(19) United States
(12) Patent Application Publication
(10) Pub. No.: US 2025/0268533 A1
(43) Pub. Date: Aug. 28, 2025

(54) INTEGRATED TRANSMITTER UNIT AND SENSOR INTRODUCER MECHANISM AND METHODS OF USE

(71) Applicant: ABBOTT DIABETES CARE INC., Alameda, CA (US)

(72) Inventor: Gary Ashley Stafford, Hayward, CA (US)

(21) Appl. No.: 18/977,334

(22) Filed: Dec. 11, 2024

Related U.S. Application Data
(63) Continuation of application No. 16/906,466, filed on Jun. 19, 2020, now abandoned.
Application No. 16/906,466 is a continuation of application No. 16/228,932, filed on Dec. 21, 2018, now Pat. No. 11,457,869.
Application No. 16/228,932 is a continuation of application No. 13/022,616, filed on Feb. 7, 2011, now Pat. No. 10,194,863.
Application No. 13/022,616 is a continuation of application No. 11/240,257, filed on Sep. 30, 2005, now Pat. No. 7,883,464.

(51) Int. Cl. A61B 5/00; A61B 5/145; G16H 10/60; G16H 40/63; G16H 50/30

(52) U.S. Cl. CPC A61B 5/6828; A61B 5/14532; A61B 5/6849; G16H 10/60; G16H 40/63; G16H 50/30; A61B 5/0002; A61B 5/6824

(57) ABSTRACT

Method and apparatus for providing an integrated transmitter unit and sensor insertion mechanism is provided.

### RELATED APPLICATIONS

The present application is a continuation of U.S. patent application Ser. No. 16/906,466, filed Jun. 19, 2020, which is a continuation of U.S. patent application Ser. No. 16/228,932, filed Dec. 21, 2018, now U.S. Pat. No. 11,457,869, which is a continuation of U.S. patent application Ser. No. 13/022,616, filed Feb. 7, 2011, now U.S. Pat. No. 10,194,863, which is a continuation of U.S. patent application Ser. No. 11/240,257, filed Sep. 30, 2005, now U.S. Pat. No. 7,883,464, all of which are incorporated herein by reference in their entireties for all purposes.

### BACKGROUND

Continuous glucose monitoring systems generally include a sensor such as a subcutaneous analyte sensor, at least a portion of which is configured for fluid contact with biological fluid, for detecting analyte levels such as for example glucose or lactate levels, a transmitter (such as for example an RF transmitter) in communication with the sensor and configured to receive the sensor signals and to transmit them to a corresponding receiver unit by for example, using RF data transmission protocol. The receiver may be operatively coupled to a glucose monitor that performs glucose related calculations and data analysis.

The transmitter may be mounted or adhered to the skin of a patient and also in signal communication with the sensor. Generally, the sensor is configured to detect the analyte of the patient over a predetermined period of time, and the transmitter is configured to transmit the detected analyte information over the predetermined period of time for further analysis. To initially deploy the sensor so that the sensor contacts and electrodes are in fluid contact with the patient's analyte fluids, a separate deployment mechanism such as a sensor inserter or introducer is used. Moreover, a separate base component or mounting unit is provided on the skin of the patient so that the transmitter unit may be mounted thereon, and also, to establish signal communication between the transmitter unit and the analyte sensor.

As discussed above, the base component or mounting unit is generally adhered to the skin of the patient using an adhesive layer that is fixedly provided on the bottom surface of the base component or the mounting unit for the transmitter.

To minimize data errors in the continuous or semi-continuous monitoring system, it is important to properly insert the sensor through the patient's skin and securely retain the sensor during the time that the sensor is configured to detect analyte levels. In addition to accurate positioning of the sensor through the skin of the patient, it is important to minimize the level of pain associated with the insertion of the sensor through the patient's skin.

Additionally, for the period of continuous or semi-continuous monitoring which can include, for example, 3 days, 5 days or 7 days, it is important to have the transmitter unit securely mounted to the patient, and more importantly, in proper contact with the analyte sensor so as to minimize the potential errors in the monitored data. Indeed, when mounted onto the skin using adhesives, bodily fluid such as sweat and muscle flexure may weaken the adhesive securing the transmitter unit onto the skin surface, and which may potentially cause the transmitter unit to detach from the skin prematurely.

In view of the foregoing, it would be desirable to have methods and apparatuses which would minimize the number of components that are needed for the patient to manipulate in order to deploy the sensor and the transmitter unit to properly be initialized and set-up so that the sensor maybe configured to monitor a biological fluid to detect, for example, analyte levels of the patient and the transmitter unit may be configured to transmit data associated with the detected analyte levels of the patient. Further, it would be desirable to have methods and apparatuses that include an integrated sensor insertion mechanism and transmitter mount or housing portion which may be mounted on the patient's skin securely, with ease and relatively little pain to the patient.

### SUMMARY OF THE INVENTION

In certain embodiments, there is provided a method and apparatus for providing an integrated transmitter unit and sensor insertion mechanism. Embodiments may include a base portion (e.g., a flexible base portion) for securing the transmitter unit around a body part of the patient, such as the patient's arm or leg and including a detachable disposable sensor introducer providing a low profile integrated data monitoring system.

In this manner, in accordance with the various embodiments of the invention, there is provided an integrated transmitter unit and sensor insertion mechanism that is configured for a multi-use disposable monitoring components for use in continuous glucose monitoring systems. More specifically, several components such as the transmitter unit, the sensor, sensor insertion mechanism (including, for example, a pre-assembled sensor, introducer and protective housing combination) and the skin mounting units are integrated into fewer components (two or less, for example), to simplify the use thereof and also to provide additional ease of use to the patients.

Moreover, an integrated transmitter unit and sensor insertion mechanism of the subject invention may be configured to depart from the reliance upon an adhesive patch as the primary method of transmitter attachment to the patient so that the mounting of the transmitter to the patient is not substantially affected by the patient's bodily fluids, cosmetics, lotions, body hair, skin type/condition or any other factor that may potentially weaken the adhesive condition of an adhesive patch.

### BRIEF DESCRIPTION OF THE DRAWINGS

FIGS. 1A, 1B and 1C illustrate a top view, a bottom view, and a perspective view, respectively, of the integrated transmitter unit and sensor insertion mechanism for use in the data monitoring system in accordance with one embodiment of the present invention;

FIG. 2 illustrates a sensor introducer unit positioned relative to the integrated transmitter and sensor insertion mechanism in pre-deployment position in accordance with one embodiment of the present invention;

FIGS. 3A-3B illustrate a front planar view and a perspective view, respectively, of the sensor introducer mechanism in pre-deployment position in accordance with one embodiment of the present invention;

FIGS. 4A-4B illustrate a front planar view and a perspective view, respectively, of the sensor introducer mechanism in deployed position in accordance with one embodiment of the present invention;

FIGS. 5A-5B illustrate front planar view and a perspective view, respectively, of the sensor introducer mechanism with the deployed sensor and the introducer in removal position, in accordance with one embodiment of the present invention;

FIG. 6 illustrates the sensor introducer unit positioned relative to the integrated transmitter and sensor insertion mechanism in post sensor deployment position in accordance with one embodiment of the present invention;

FIGS. 7A-7C illustrate a front planar view, a perspective view, and a side planar view, respectively of the deployed sensor with the introducer removed in accordance with one embodiment of the present invention;

FIG. 8 illustrates an introducer assembly for use in angled sensor insertion in accordance with one embodiment of the present invention;

FIG. 9 illustrates a sensor introducer assembly positioned relative to the integrated transmitter and sensor insertion mechanism in pre-deployment position for angled insertion in accordance with one embodiment of the present invention;

FIG. 10 illustrates the sensor introducer unit positioned relative to the integrated transmitter and sensor insertion mechanism in post sensor deployment position for angled insertion in accordance with one embodiment of the present invention; and

FIGS. 11A-11B illustrate a perspective view and side planar view, respectively of the deployed sensor with the introducer removed for angled sensor insertion in accordance with one embodiment of the present invention.

### DETAILED DESCRIPTION

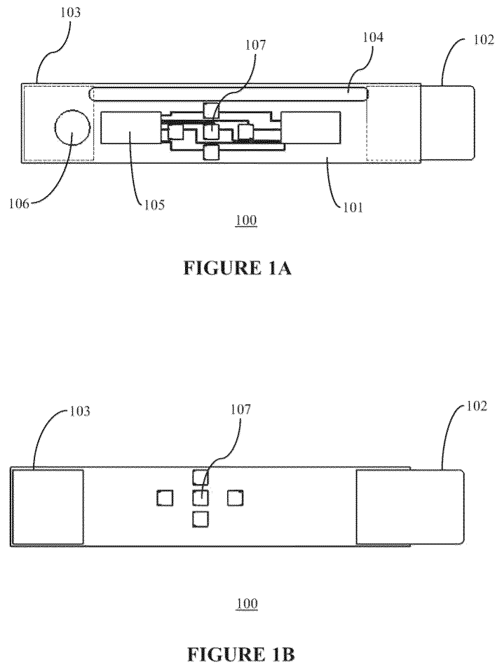

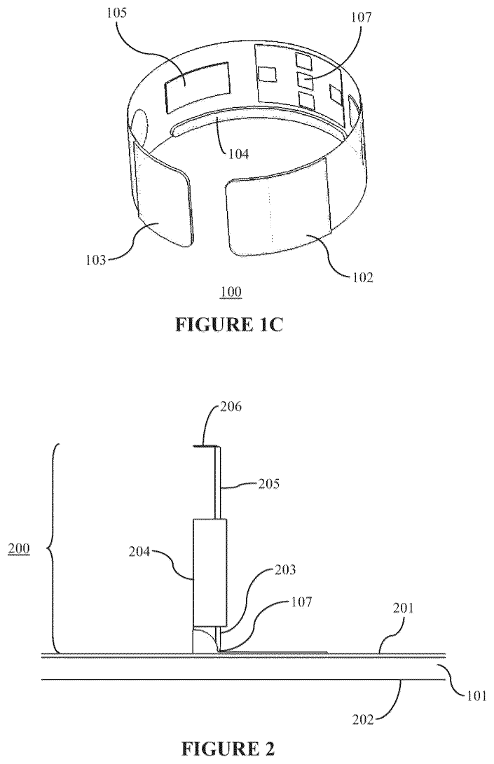

FIGS. 1A, 1B and 1C illustrate a top view, a bottom view, and a perspective view, respectively, of an integrated transmitter unit and sensor insertion mechanism for use in a data monitoring system in accordance with one embodiment of the present invention. Referring to FIGS. 1A-1C, an integrated transmitter unit and sensor insertion mechanism 100 in one embodiment includes a substantially elongated and base portion 101, which may be a flexible base portion. The base portion 101 is substantially shaped and configured in one embodiment to be of sufficient length to partially or completely wrap around the patient's arm, wrist, thigh, calf, torso or any other part of the patient's body where the sensor is to be positioned and introduced. In one embodiment, the base portion 101 may be made of a material that is pre-formed or molded by the patient, to follow the curvature of the anatomy, or may be made of a flexible form filling or conformable material such as fabric, strap or sleeve.

Referring back to FIGS. 1A-1C, in one embodiment of the present invention, there is provided a securing mechanism that includes parts 102 and 103 to the respective ends of the base portion 101 as shown in the Figure, which may be in the form of hook portion 102 and a loop portion 103. More specifically, in the embodiment including a Velcro-type securing mechanism, the hook portion 102 provided at a first end of the base portion 101 includes a Velcro hook which is configured to be mated with the Velcro loop at the loop portion 103 at the second end of the base portion 101. In this manner, the integrated transmitter unit and sensor insertion mechanism 100 may be securely and substantially fixedly positioned around the patient's arm, for example, using the Velcro mechanism provided thereon. Within the scope of the present invention, the hook portion 102 and the loop portion 103 may comprise any other suitable securing mechanism, including but not limited to, a buckle type securing system, a button type securing system, a hook or latch mechanism, and a zipper type fastening mechanism.

Referring again to FIGS. 1A-1C, the integrated transmitter unit and sensor insertion mechanism 100 in one embodiment is provided with the transmitter electronics 105 substantially embedded within the base material 101. Moreover, the transmitter electronics 105 is also configured to be in electrical communication with a transmitter antenna 104 (for example, an RF transmission antenna), as well as a power source 106 (for example, a disposable battery) that may also be provided substantially within the base material 101. In one embodiment, the integrated transmitter unit and sensor insertion mechanism 100 may be provided to the patient fully assembled with the power source 106, the transmitter antenna 104, and the transmitter electronics 105 embedded or laminated within the layers of the base material 101. In this manner, once positioned, the transmitter unit and sensor insertion mechanism 100 may be worn by the patient and may have a very low profile, with for example, approximately, 4-5 mm of thickness so as to advantageously minimize physical hindrance to the patient's daily activities while using the transmitter unit and sensor insertion mechanism 100. However, other dimensions are possible as well.

Referring still again to FIGS. 1A-1C, there is also shown a sensor insertion location 107 provided on the base material 101 of the integrated transmitter unit and sensor insertion mechanism 100. In this manner, during the insertion process, the patient is able to readily determine the proper location of sensor insertion so as to accurately and effectively deploy the sensor to be in signal communication with the transmitter unit electronics (for example, the corresponding contact points for the respective electrodes such as the working, reference, and counter (or reference/counter) electrodes of the sensor, and optionally, the guard trace of the sensor).

In the manner described above, in accordance with one embodiment of the present invention, the integrated transmitter unit and sensor insertion mechanism 100 is configured to depart from the reliance upon an adhesive patch as the primary method of transmitter attachment to the patient so that it is not substantially affected by the patient's bodily fluids, cosmetics, lotions, body hair, skin type/condition or any other factor that may potentially weaken the adhesive condition of the adhesive patch. Moreover, in one embodiment, the base material 101 of the integrated transmitter unit and sensor insertion mechanism 100 may be formulated to provide some measure of moisture vapor transmission rate (MVTR) to allow the patient's skin to breath or ventilate.

FIG. 2 illustrates a sensor introducer unit positioned relative to the integrated transmitter and sensor insertion mechanism in pre-deployment position in accordance with one embodiment of the present invention. Referring to FIG. 2, in one embodiment of the present invention, a sensor introducer assembly 200 is provided and includes an introducer mechanism 205 including a handle portion 206 configured for manipulation during manual (or otherwise) sensor insertion process. Also provided in the introducer mechanism 205 is a sensor casing 204 that is operatively coupled to the introducer mechanism 205 and which is configured to house a sensor (not shown) in physical cooperation with the introducer mechanism 205.

Referring back to FIG. 2, it can be seen that a tip portion 203 of the introducer mechanism 205 is substantially aligned with the sensor insertion location 107 on a top surface 201 of the base portion 101 of the integrated transmitter unit and sensor insertion mechanism 100. Moreover, it can be further seen from FIG. 2 that a bottom surface 202 of the base portion 101 of the integrated transmitter unit and sensor insertion mechanism 100 is in physical contact with the skin of the patient.

As will be described in further detail below, upon positioning the tip portion 203 of the introducer mechanism 205 substantially aligned with the sensor insertion position 107 on the base portion 101, the patient may depress upon the handle portion 206 of the introducer mechanism 205 so as to insert the analyte sensor transcutaneously such that at least a portion of the sensor is positioned to be in fluid contact with the patient's biological fluids, such as for example, interstitial fluids. Other manners of activating the introducer may be used within the scope of the present invention.

Thereafter, upon insertion and positioning the sensor, the handle portion 206 may be collapsed into the sensor casing 204, and the sensor casing 204 pivoted approximately 90 degrees substantially about the sensor insertion position 107, so as to collapse the sensor casing 204 onto the upper surface 201 of the base portion 101 of the integrated transmitter unit and sensor insertion mechanism 100 including the sensor electrical contact surface. In this manner, a low profile, substantially thin sensor and transmitter combination may be provided to the patient to be worn for a predetermined period of time, while minimizing potential interference with the patient's daily physical activities and securely holding the transmitter in place on the patient.

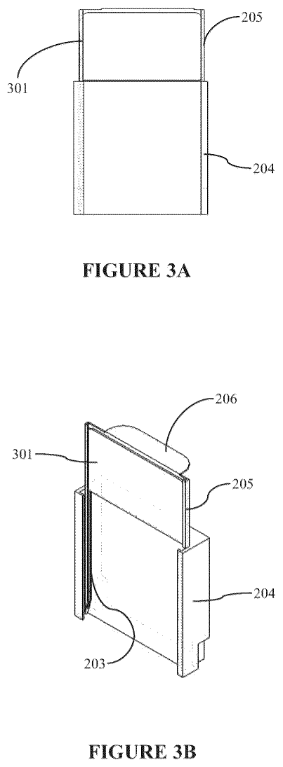

FIGS. 3A-3B illustrate a front planar view and a perspective view, respectively, of the sensor introducer mechanism in pre-deployment position in accordance with one embodiment of the present invention. Referring to FIGS. 3A-3B, it can be seen that the sensor 301 is provided substantially engaged with the introducer 205, a portion of each of which is guided within and through the inner section of the sensor casing 204. Moreover, the introducer tip portion 203 is configured to substantially contain the portion of the sensor 301 that is to be placed under the patient's skin, e.g., subcutaneously.

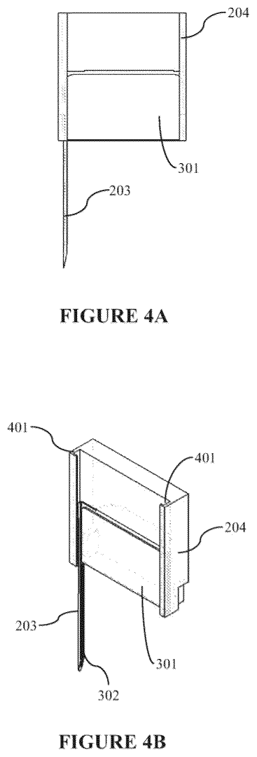

FIGS. 4A-4B illustrate a front planar view and a perspective view, respectively, of the sensor introducer mechanism in deployed position in accordance with one embodiment of the present invention. Referring to FIGS. 4A-4B, upon manual operation of the introducer handle portion 206, by the force applied by the patient thereonto, the introducer tip portion 203 is configured to guide a sensor tip 302 of the sensor 301 through the skin of the patient, so as to position at least a portion of the sensor tip 302 in fluid contact with the patient's biological fluids such as interstitial fluid. Moreover, it can be seen from FIG. 4B that the sensor casing 204 in one embodiment may be provided with one or more inner grooves 401 which are configured to substantially guide the movement of the introducer 205 and the sensor 301 through the sensor casing 204, and further, to provide substantially fixed support of the introducer 205 position relative to the sensor insertion position 107 during the operation of the introducer handle portion 206 to transcutaneously deploy the sensor 301.

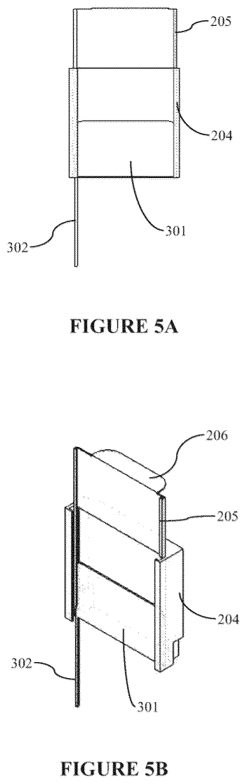

FIGS. 5A-5B illustrate a front planar view and a perspective view, respectively, of the sensor introducer mechanism with the deployed sensor and the introducer in removal position, in accordance with one embodiment of the present invention. Referring to FIGS. 5A-5B, it can be seen that the handle portion 206 and the introducer 205 are protruded out of the sensor casing 204 upon sensor deployment.

More specifically, in one embodiment, after the patient applies pressure onto the handle portion 206 so as to deploy the sensor 301, the patient may retract or withdraw the introducer 205 from the deployed position, by retracting or pulling the handle portion 206 in the opposite direction of the sensor deployment direction. Alternatively, the introducer 205 may be provided with a spring bias mechanism or a similar mechanism which would allow the introducer 205 to be retracted substantially automatically after deployment of the sensor 301.

Referring back to FIGS. 5A-5B, after deployment of the sensor 301 and retraction of the introducer 205, the introducer 205 may be detached from the sensor casing 204 and discarded. Alternatively, the introducer 205 in a further embodiment may be configured and sized so as to be substantially and completely housed within the sensor casing 204 upon deployment of the sensor 301.

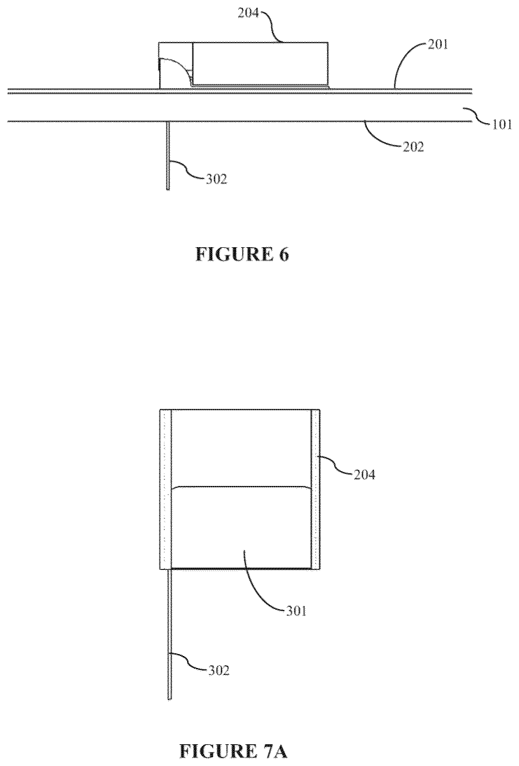

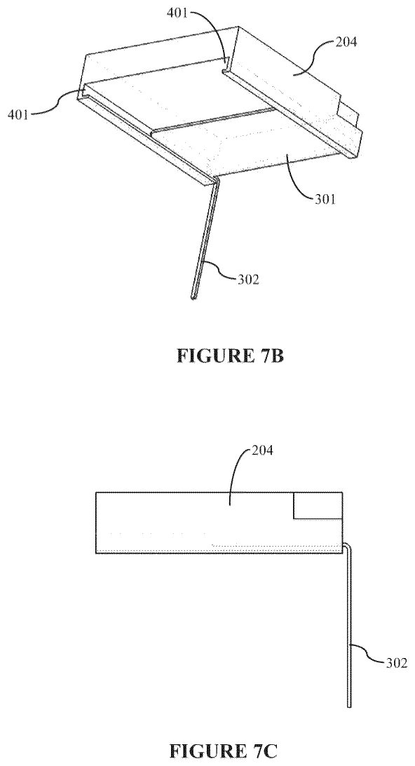

FIG. 6 illustrates the sensor introducer unit positioned relative to the integrated transmitter and sensor insertion mechanism in post sensor deployment position in accordance with one embodiment of the present invention. Moreover, FIGS. 7A-7C illustrate a front planar view, a perspective view, and a side planar view, respectively of the deployed sensor with the introducer removed in accordance with one embodiment of the present invention. Referring to the Figures, it can be seen that the sensor casing 204 may be provided in a locked and secure position on the upper surface 201 of the base portion 101 of the integrated transmitter unit and sensor insertion mechanism 100 such that a substantially low profile, sensor and transmitter system may be provided for extended wear and use by the patients to monitor one or more biological fluids and for transmission of data associated with the detected or monitored fluid to a receiver unit for further data processing, health management diagnosis and treatment.

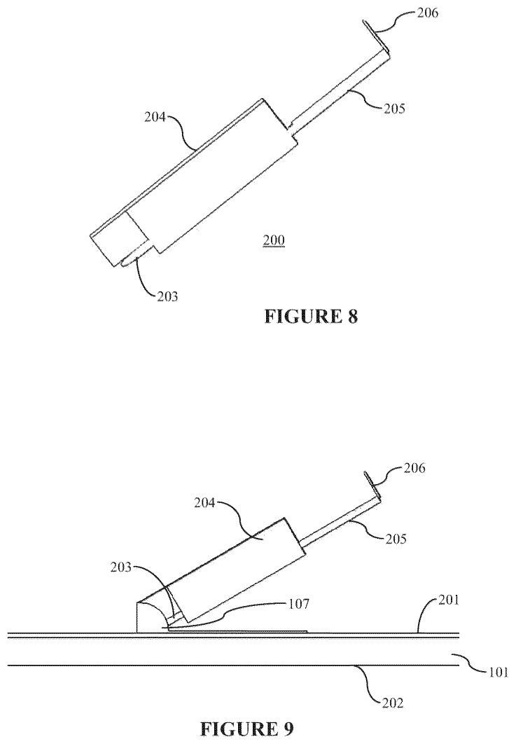

FIG. 8 illustrates an introducer assembly for used in angled sensor insertion in accordance with one embodiment of the present invention, and FIG. 9 illustrates the sensor introducer assembly positioned relative to the integrated transmitter and sensor insertion mechanism in pre-deployment position for angled insertion in accordance with one embodiment of the present invention. It can be seen that for applications where the insertion is desirable at an angle other than about 90 degrees relative to the surface of the patient's skin, within the scope of the present invention, the sensor introducer assembly 200 may be configured to provide angled insertion for varying angles of sensor insertion, and which in turn, may correspondingly vary the depth at which the sensor tip 302 is positioned below the patient's skin and in fluid contact with the patient's biological fluids such as interstitial fluid in the subcutaneous space, or the like.

For example, it can be seen that given a substantially fixed length of the sensor tip 302, the greater the angle of the insertion (relative to the surface of the patient's skin), the deeper the position of the sensor tip 302 placed under the skin, and where the deepest position of subcutaneous placement of the sensor tip 302 is achieved substantially at about 90 degrees relative to the surface of the patient's skin, and as illustrated above in conjunction with the embodiment shown in FIG. 6.

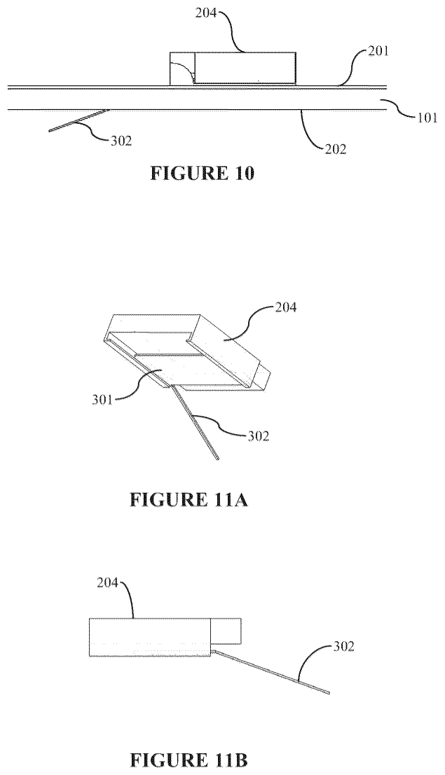

Additionally, FIG. 10 illustrates the sensor introducer unit positioned relative to the integrated transmitter and sensor insertion mechanism in post sensor deployment position for angled insertion, and FIGS. 11A-11B illustrate a perspective view and a side planar view, respectively, of the deployed sensor with the introducer removed for angled sensor insertion in accordance with one embodiment of the present invention.

As discussed above, in accordance with the various embodiment of the present invention, the base portion 101 is placed on the patient such as around the patient's arm and secured in position using the securing mechanism, e.g., hook portion 102 and the loop portion 103. Within the scope of the present invention, other methods and devices for securing the base portion 101 may be used such as, for example, metal hooks, medical grade adhesive tape, elastic bands, or straps with buckles or any other equivalent methods or devices for securing the base portion 101 onto the patient.

The sensor casing 204 with the introducer 205 and the sensor 301, pre-assembled therein, may be placed on the base portion 101 so as to substantially align the introducer tip 203 with the sensor insertion position 107 of the base portion 101. While one sensor insertion position 107 is shown in the Figures, within the scope of the present invention, a plurality of sensor insertion positions may be provided on the upper surface 201 of the base portion 101. This would provide the additional convenience for the patients by providing several insertion site locations on the skin of the patient after the base portion 101 is substantially fixedly positioned on the patient's skin. Additionally, within the scope of the present invention, the base portion may be provided with a plurality of sensor insertion positions 107 such that the integrated transmitter unit and sensor insertion mechanism 100 may be configured for use with multiple sensors either concurrently or sequentially.

In one aspect of the present invention, the transmitter electronics 105 may be configured to selectively shut off or disable the electronics at the sensor insertion positions after the sensor 301 is in use for the prescribed period of time (for example, about 1 day, about 3 days, about 5 days, about 7 days or more, e.g., about 30 days or more). In this manner, it is possible to provide the additional safety precaution by preventing continued used of the integrated transmitter unit and sensor insertion mechanism 100 after a specified number of uses (corresponding to the number of the sensor insertion positions). This option may prevent the patients from multiple usage of the same sensor 301 rather than discarding after the initial usage, and also, for using the integrated transmitter unit and sensor insertion mechanism 100 beyond the recommended periods of usage frequency. Additionally, within the scope of the present invention, the power supply 106 may be provided with a low capacitor disposable battery so as to limit the life of the integrated transmitter unit and sensor insertion mechanism 100 once it has been activated.

In addition, within the scope of the present invention, the insertion process of the sensor 301 is described as performed manually by the patient. Alternatively, within the scope of the present invention, the insertion process of the sensor 301 may be configured with a semi-automated mechanism or a fully automated mechanism provided with an insertion trigger switch, for example. Moreover, within the scope of the present invention, angled insertion of the sensor 301 may be achieved by the design and orientation of the sensor casing 204 at the sensor insertion position 107.

Additionally, within the scope of the present invention, the physical dimensions of the sensor casing 204 and the orientation of the introducer 205 in cooperation with the sensor casing 204 may provide the desired sensor insertion depth, and also to control the ease of sensor deployment. Indeed, when the introducer 205 bottoms out at the sensor insertion position 107 within the sensor casing 204, the sensor depth or the below-the-skin position is determined to be reached, and thereafter, the introducer 205 may be safely discarded, for example, by detaching from the sensor casing 204 or substantially completely encasing within the sensor casing 204. Then, the sensor casing 204 may be rotatably pushed substantially about the sensor insertion position 107 so that it may be maintained in a locked position, thus holding the sensor 301 in place, and establishing electrical communication with the transmitter electronics 105 laminated, for example, within the base portion 101 of the integrated transmitter unit and sensor insertion mechanism 100. In one embodiment, the sensor casing 204 may be provided with a compressible seal around its perimeter to prevent moisture, particulate, and other foreign materials from contaminating the transmitter electronics 105 or potentially compromising the integrity of the electrical contacts and signal.

In this manner, the detected analyte levels from the sensor 301 may be provided to transmitter electronics 105, which is, in one embodiment, configured to wirelessly transmit data corresponding to the detected analyte levels from the sensor to a receiver unit via the antenna 104, where the receiver unit may include a glucose monitor unit and/or an insulin pump unit and/or a computer terminal, or any other electronic device capable of being configured for wireless (or other) communication. Within the scope of the present invention, the receiver unit functions may be integrated into portable electronic devices such as a watch, a pager, a mobile telephone, personal digital assistant, and the like. Additional information on the detection, monitoring and analysis of analyte levels are described in further detail in U.S. Pat. No. 6,175,752 entitled “Analyte Monitoring Device and Methods of Use” the disclosure of which is incorporated herein by reference for all purposes.

In a further embodiment, the transmitter electronics 105 may include a wireless communication unit for wireless transmission of the signal, where the wireless communication unit may include one or more of a radio frequency (RF) communication unit, a Bluetooth® communication unit, an infrared communication unit, an 801.11x communication unit, or a Zigbee® communication unit. Similarly, the receiver unit may be configured to support one more or of the above-referenced wireless communication protocols to communicate with the transmitter unit.

In this manner, within the scope of the present invention, there is provided an integrated transmitter unit and sensor insertion mechanism that is configured for a multi-use disposable monitoring component for use in continuous glucose monitoring systems. More specifically, several components such as the transmitter unit, the sensor, sensor insertion mechanism (including, for example, a pre-assembled sensor, introducer and protective housing combination) and the skin mounting units are integrated into fewer components (two or less, for example), to simplify the use and also to provide additional ease of use to the patients.

Accordingly, within the scope of the present invention, it is possible to eliminate the separate system components of the skin attachment system, sensor insertion and the transmitter unit along with the associated safety precautions, material costs, weight, packaging, handling, disposal, and attaching to the skin patch. Furthermore, a low profile integrated system may be provided that would substantially minimize potential interference with the patient's normal daily activities. In the embodiments described above, while the handle portion 206 (FIG. 2) for example, is configured to be discarded after the sensor insertion process, within the scope of the present invention, the handle portion of the sensor introducer assembly may be configured to be integrated within the sensor introducer assembly—for example, within the sensor casing 204.

Moreover, the integrated transmitter unit and sensor insertion mechanism 100 may be configured to depart from the reliance upon an adhesive patch as the primary method of transmitter attachment to the patient so that it is not substantially affected by the patient's bodily fluids, cosmetics, lotions, body hair, skin type/condition or any other factor that may potentially weaken the adhesive condition of the adhesive patch. Moreover, in one embodiment, the base material 101 of the integrated transmitter unit and sensor insertion mechanism 100 may be formulated to provide some measure of moisture vapor transmission rate (MVTR) to allow the patient's skin to breath or ventilate.

In the manner described above, in accordance with one embodiment, there is provided an integrated sensor and transmitter device including a base unit configured for mounting onto a skin of a patient, a transmitter unit integrally provided in the base unit, a sensor assembly disposed on a surface of the base unit, the sensor assembly including a sensor configured to couple to the transmitter unit.

The base unit may be flexible, and further, may include a first end segment and a second end segment, the first end segment configured to couple to the second end segment so as to retain the base unit on the skin of the patient. Moreover, in one embodiment, the first end segment may include a Velcro hook and the second end segment may include Velcro latch.

The transmitter unit in one embodiment may include flexible electronic circuitry, where in certain embodiments the electronic circuitry may be laminated into the base unit.

Additionally, the transmitter unit may be configured to substantially conform to the shape of the base unit.

The sensor assembly in one embodiment may include an introducer coupled to at least a portion of the sensor, the introducer configured to transcutaneously position at least the portion of the sensor.

The introducer may be configured to transcutaneously position at least the portion of the sensor to be in fluid contact with a biological fluid of the patient, where the biological fluid of the patient includes interstitial fluid or blood of the patient.

Further, the introducer may be configured to be detachably removed from the sensor assembly after at least the portion of the sensor is transcutaneously positioned.

Additionally, the introducer may be configured to transcutaneously position at least the portion of the sensor at an angle other than about 90 degrees relative to the surface of the patient's skin.

In a further embodiment, the sensor assembly may be configured to substantially pivot onto the base unit so that the sensor is in signal communication with the transmitter unit.

Moreover, the sensor in one embodiment is an analyte sensor. The sensor may be configured to detect any analyte such as glucose, lactate, etc. Additional analytes that may be determined include, for example, acetyl choline, amylase, bilirubin, cholesterol, chorionic gonadotropin, creatine kinase (e.g., CK-MB), creatine, DNA, fructosamine, glucose, glutamine, growth hormones, hormones, ketones, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be determined. A sensor may be configured to detect two or more analytes such as two or more analyte mentioned herein.

An apparatus in a further embodiment of the present invention includes a flexible base unit including an outer surface and an inner surface, said inner surface configured to contact a skin of a patient, a sensor assembly including a sensor disposed in the sensor assembly, and an introducer substantially provided in the sensor assembly to couple to at least a portion of the sensor; the introducer configured to position at least a portion of the sensor transcutaneously through the skin of the patient, and a transmitter unit integrally disposed in the base unit, the transmitter unit configured to receive one or more signals from the sensor.

The introducer may be configured to be removably detached from the sensor assembly after at least the portion of the sensor is transcutaneously positioned.

Moreover, the one or more signals from the sensor may substantially correspond to a respective one or more analyte levels of the patient.

Additionally, the transmitter unit may be configured to wirelessly (or otherwise) transmit data corresponding to the one or more signals received from the sensor, where a receiver unit may be additionally provided and configured to receive data from the transmitter unit, where the received data corresponds to one or more analyte levels of the patient. Either or both of the transmitter or receiver may be a transceiver.

The base unit in one embodiment may be configured to be securely attached substantially around one of an arm, a torso, a thigh, a calf, a waist or wrist of the patient. In certain embodiments, the base unit is flexible.

A method in accordance with still another embodiment of the present invention includes the steps of securing a transmitter unit substantially around a body part of a patient, and introducing at least a portion of a sensor through the skin of the patient so that the portion of the sensor is in fluid contact with a biological fluid of the patient, and further, where the sensor is in electrical contact with the transmitter unit.

A system in accordance with yet a further embodiment of the present invention includes an integrated housing including a transmitter unit and a sensor; the integrated housing configured for mounting onto a skin of a patient, and a sensor introducer assembly mounted onto the integrated housing, the introducer assembly configured to position at least a portion of the sensor under the skin of the patient, where the transmitter unit is in electrical contact with the sensor, and configured to transmit one or more signals corresponding to a respective one or more signals received from the sensor.

In another embodiment, the system may further include a receiver unit configured to receive the one or more signals transmitted by the transmitter unit, where the receiver unit may include one of an infusion pump, a monitoring device, a personal digital assistant, a pager, or a mobile telephone.

Various other modifications and alterations in the structure and method of operation of this invention will be apparent to those skilled in the art without departing from the scope and spirit of the invention. Although the invention has been described in connection with specific preferred embodiments, it should be understood that the invention as claimed should not be unduly limited to such specific embodiments. It is intended that the following claims define the scope of the present invention and that structures and methods within the scope of these claims and their equivalents be covered thereby.

1-28. (canceled)

29. A wearable assembly for monitoring a glucose level of a user, the wearable assembly comprising:
a base portion;
electronics substantially housed within the base portion, the electronics comprising a power source and transmitter electronics coupled with an antenna; and
a securing mechanism comprising a first part and a second part, wherein the first part is coupled with a first end of the base portion and the second part is coupled with a second end of the base portion,
wherein the electronics are coupled with a plurality of sensing elements configured to measure the glucose level of the user, and wherein the transmitter electronics are configured to wirelessly transmit data indicative of the glucose level via the antenna to a receiver unit,
wherein the plurality of sensing elements are configured to contact a skin layer of the user, wherein the plurality of sensing elements are disposed on a side of the base portion which is configured to face the skin layer of the user, wherein the plurality of sensing elements are arranged in an array configured to at least partially disposed around a central aperture and
wherein at least a portion of the wearable assembly is configured to wrap around an appendage of the user.

30. The assembly of claim 29, wherein the base portion comprises a substantially elongated shape.

31. The assembly of claim 29, wherein the first part of the securing mechanism comprises a hook portion and the second part of the securing mechanism comprises a loop portion.

32. The assembly of claim 29, wherein the securing mechanism comprises one or more of a buckle, a hook, a latch, a button, or a zipper.

33. The assembly of claim 29, wherein at least a portion of the electronics are embedded or laminated within one or more layers of the base portion.

34. The assembly of claim 29, wherein the one or more sensing elements are in signal communication with the electronics coupled to the base portion.

35. The assembly of claim 29, wherein the wearable assembly is further configured to attach with the appendage without use of an adhesive.

36. The assembly of claim 29, wherein the transmitter electronics are configured to wirelessly transmit one or more signals to a receiver unit.

37. The assembly of claim 36, wherein the receiver unit is a mobile phone.

38. The assembly of claim 36, wherein the receiver unit is an insulin pump.

39. The assembly of claim 29, wherein the base portion comprises a flexible material configured to substantially conform to the shape of the appendage of the user.

40. The assembly of claim 29, wherein the antenna is disposed on a side of the base portion which is configured to face the skin layer of the user.

41. The assembly of claim 29, wherein the central aperture and the plurality of sensing elements are disposed on a side of the base portion which is configured to face the skin layer of the user.

42. The assembly of claim 29, wherein the first part of the securing mechanism is configured to engage with the second part of the securing mechanism such that the wearable assembly is configured to be securely wrapped around the appendage of the user.

43. The assembly of claim 29, wherein the appendage is a wrist or an arm.

44. The assembly of claim 29, wherein the array of sensing elements is configured to be positioned in a radial arrangement around the central aperture.

45. The assembly of claim 29, wherein the transmitter electronics are configured to transmit the data indicative of the glucose level according to a Bluetooth communication protocol.
